(19) United States
(12) Patent Application Publication
(10) Pub. No.: US 2021/0338424 A1
(43) Pub. Date: Nov. 4, 2021

(54) DELIVERY SYSTEM FOR IMPLANTS USED IN STRUCTURAL HEART DISEASES BY A MINIMALLY INVASIVE METHOD

(71) Applicant: Innovations for Heart and Vessels Sp. z o.o., Katowice (PL)

(72) Inventors: Pawel Buszman, Katowice (PL); Piotr Dobrzynski, Zabrze (PL); Janusz Kasperczyk, Katowice (PL); Michal Sobota, Czestochowa (PL); Katarzyna Jelonek, Czestochowa (PL); Jakub Wlodarczyk, Poronin (PL); Mateusz Stojko, Laziska Gorne (PL); Mariusz Pawlak, Zabrze (PL); Wojciech Klein, Knurow (PL); Jacek Gnilka, Gliwice (PL); Arkadiusz Mezyk, Gliwice (PL); Marian Zembala, Tarnowskie Gory (PL); Michal Zembala, Zbroslawice (PL); Joanna Sliwka, Zabrze (PL); Krzysztof Milewski, Katowice (PL); Piotr Buszman, Katowice (PL); Piotr Hirnle, Warszawa (PL); Jerzy Nozynski, Zabrze (PL)

(21) Appl. No.: 17/262,499

(22) PCT Filed: Jul. 26, 2018
(86) PCT No.: PCT/PL2018/050039

§ 371 Date: Jan. 22, 2021

(30) Foreign Application Priority Data
Jul. 24, 2018 (PL) P.426433

(51) Int. Cl. A61F 2/24

(52) U.S. Cl. CPC A61F 2/2433; A61F 2/2436

(57) ABSTRACT

A delivery system for implants used in structural heart diseases by a minimally invasive method comprises a catheter (2) which is connected with the distal section of the balloon (1) and on the other side with the straight cap with the lateral canal (10) to which a pressure syringe for pumping the balloon is attached. The catheter is covered from the outside with a special shield (8). The balloon (1) has a necking in the middle section and inside the balloon and in the catheter (8) there is a canal (6) led outside the balloon formed at the end in the shape of a coil (3). Inside the balloon (1) or outside it at its distal and proximal ends the system is equipped with at least one anchoring mechanism shapely formed crimped on the balloon surface of the implant preventing its uncontrolled moving.

The invention relates to the delivery system for implants used in structural heart diseases by a minimally invasive method intended for transcatheter treatment of heart valve defects. The solution presented below relates to medical devices used to deliver instruments and devices (prostheses, implants) used for treatment of heart structural diseases by a minimally invasive method.

The above mentioned delivery system is used to insert a medical device (a prosthesis, an implant) intended for treatment of heart valve defects in an affected area in a safe manner. Additionally, use of this system allows precise adjustment of the location of the device or the implant (positioning) used for treatment of valve defects. It also secures the delivered device against damages. Furthermore, the system enables maneuvering and ensures protection against sliding or improper positioning.

Currently the gold standard of treatment severe aortic stenosis is still a surgical heart valve replacement for biological or artificial prosthetic heart valve. However, the growing number of elderly patients who are admitted to hospital for treatment, as well as frequent occurence of concomitant afflictions is the reason why ⅓ of patients after 75 years old cannot be qualified for surgery because of the surgical risk which is too high. When Alain Cribier performed the first Tavi procedure (transcatheter aortic valve implantation) in 2002 the alternative for a classical surgery appeared. With use of a special catheter by minimally invasive access (most frequently through femoral artery) a crimped prosthetic heart valve is delivered in the place of a native aortic valve and afterwards it expands (manually with use of a balloon or of a mechanical mechanism, either a prosthetic valve opens itself by gradual removing an integument which supports it) enabling its anchoring in the aortic annulus. This method revolutionized the approach to aortic stenosis treatment, offering the possibility of therapy to the worst-case patients. Its efficacy has been confirmed in extensive clinical research (publications: M. J. Mack, M. B. Leon, C. R. Smith et al., 5-year outcomes of transcatheter aortic valve replacement or surgical aortic valve replacement for high surgical risk patients with aortic stenosis a randomised controlled trial, Lancet 385(9986) (2015) 2477-84; G. M. Deeb, M. J. Reardon, S. Chetcuti et al., U.S.C.I. CoreValve, 3-Year Outcomes in High-RiskPatients Who Underwent Surgical or Transcatheter Aortic Valve Replacement, Journal ofthe American College of Cardiology 67(22) (2016) 2565-74; L. Sondergaard, D. A. Steinbruchel, N. Ihlemann et al., Two-Year Outcomes in Patients With Severe Aortic Valve Stenosis Randomized to Transcatheter Versus Surgical Aortic Valve Replacement: The All-Comers Nordic Aortic Valve Intervention Randomized Clinical Trial, Circulation. Cardiovascular interventions 9(6) (2016); L. Sondergaard, Clinical, safetyand echocardiographic outcomes from the NOTION trial: 4 year follow-up data in allcomer patients with severe aortic valve stenosis, (2017); M. B. Leon, C. R. Smith, M. J. Mack et al., Transcatheter or Surgical Aortic-Valve Replacement in Intermediate-Risk Patients, N Engl J Med 374(17) (2016) 1609-20; M. J. Reardon, N. M. Van Mieghem, J. J. Popma et al., Surgical or Transcatheter Aortic-Valve Replacement in Intermediate-Risk Patients, N Engl J Med 376(14) (2017) 1321-1331.) not giving way to classical surgery.

As a result more and more patients who are younger and less severely ill are indicated for TAVI procedure. It also refers to patients with aortic valve improper disclosure. Despite promising results, TAVI like any other method is not free from drawbacks. The main ones are the following: a relatively large size of delivery systems causing the risk of peripheral blood vessels damage, a possibility of malpositioning of the prosthetic valve during implantation, the necessity of implantation of the stimulator due to elicited disordered conducting, the risk of coronary obstruction, and the occurence of paravalvular leakage. The above mentioned problems are thoroughly described by M. J. Reardon, N. M. Van Mieghem, J. J. Popma in Surgical or Transcatheter Aortic-Valve Replacement in Intermediate-Risk Patients, N Engl J Med 376(14) (2017) 1321-1331 and by C. Fraccaro, M. Napodano, G. Tarantini, in Conduction disorders in the setting of transcatheter aortic valve implantation: a clinical perspective, Catheterization and cardiovascular interventions: official journal of the Society for Cardiac Angiography & Interventions 81(7) (2013)1217-23. Another issue which has not been clarified so far is the durability of the said prosthetic valves due to lack of sufficient period of observations, which is addressed by M. Arsalan, T. Walther w Durability of prostheses for transcatheter aortic valve implantation, Nat Rev Cardiol 13(6) (2016) 360-7. Taking into consideration analogies to biological prosthetic heart valves implanted surgically and also bearing in mind the fact that TAVI valves are subject to bigger loads due to the process of crimping and expansion this issue may not be omitted. The above mentioned constraints necessitate carrying out further research, introducing new technologies and improving existing ones in order to improve the treatment results and the safety of patients, in particular in the situation when less severely ill patients are to undergo the treatment.

The delivery system known from the patent description US2016175559 has a canal extending between proximal and distal ends, where a balloon is disposed at the proximal end , before the pig tail” end. Behind the balloon and at the “pig tail” end there may be openings for administering medication. Such a construction of a balloon does not allow to deliver implants used for treatment of heart diseases. Additionally, there is no protrusion which prevents the implant from sliding inside the balloon.

The delivery system known from the patent description US2018071498 comprises a textile balloon which is able to press and decompress, thanks to which the shape of a balloon catheter may be changed. Such a construction is deprived of a coil at the end, which allows the protection of the vessel wall. Additionally, use of the textile material instead of plastic to form the balloon walls prevents achieving a low profile after constraint. Additionally, it may be difficult to place any implant for treatment of heart valve defects on the balloon's surface due to lack of elements preventing it from sliding.

The valvuloplasty catheter known from the patent description US2015066069 comprises a balloon for valvuloplasty. The balloon has a smaller diameter waist, and a shaft delivering a contrasting fluid for filling the balloon. Thanks to the above mentioned fact expansion is achieved. The system is deprived of an atraumatic end which may irritate and damage the vessel and cause impairment of the left heart ventricle.

The aim of the invention is to eliminate the drawbacks of the currently used delivery systems for implants used for treatment of structural heart diseases and to create a new delivery system. The system will facilitate the implantation process by possibility of free maneuvering, positioning and repositioning the implant. Additionally, it will protect an implant, a prosthesis or a device against damage during introducing to the vessel. The aim of the invention is also to construct the delivery system which does not cause trauma for the peripheral vessel.

The invention relates to the delivery system used in structural heart diseases by a minimally invasive method comprising a catheter connected with the distal section of the balloon and from the other side connected with a straight cap comprising a collateral canal to which a pressure syringe for pumping a balloon is attached. The catheter is protected from the outside by a shield. The balloon has a necking in the middle section, and inside the balloon and in the catheter there is a canal which is led outside the balloon and at the end it is formed in the shape of a coil. Inside the balloon or outside it at its distal and proximal ends the system is equipped with at least one anchoring mechanism shapely formed, crimped on the implant balloon surface, which prevents its uncontrolled movement. The anchoring mechanism is located on the straight segment of the canal at the intersection with the proximal section of the balloon and it is formed in a shape of a cone, whose extended section is directed towards the balloon. The anchoring mechanisms are also optionally made inside the balloon in its distal and proximal sections and they are formed as protrusions of the canal inside the balloon. The anchoring mechanism is also made on the proximal end of the catheter shield in the form of a cot with a diameter growing at the outlet. The anchoring mechanisms may also occur in the form of markers located on the outside surface of the canal. The diameter of the marker circumference is bigger than the diameter of the canal circumference. The anchoring system appears in the form of a cot at the end of the shield in the form of a cone dilation in the direction of the distal end of the balloon.

The advantage of the system is an increased possibility of stable and firm mounting of an implant, a prosthetic heart valve or another device used for treatment of a heart valve defect. Due to the fact that anchoring mechanisms are formed inside and outside a balloon catheter, and also due to the fact that a crimped device or a medical product is placed on the surface of a crimped balloon it is possible to prevent its uncontrolled movement while delivering it to the implantation site.

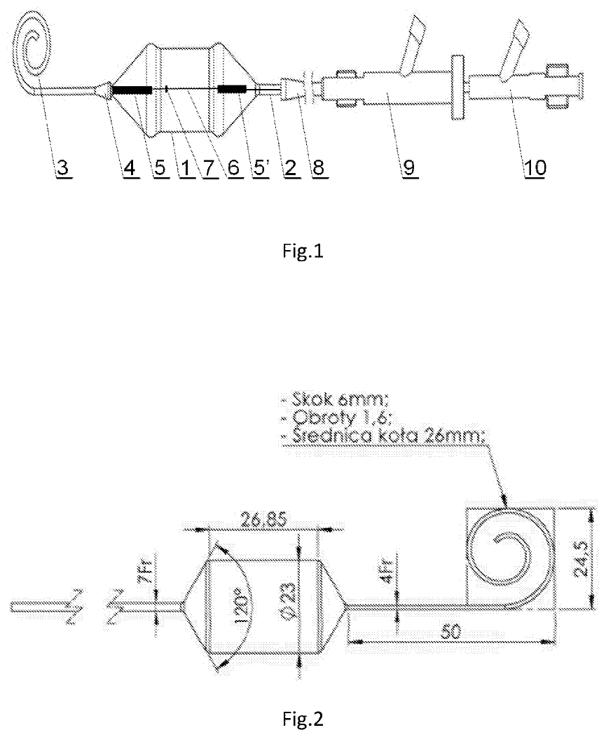

The invention is presented in the example of realisation in the drawing in which FIG. 1 shows the system from the side, and. FIG. 2 shows the example of realisation with exposed dimensions.

The said system consists of a delivering balloon 1 made of polyamide, where its shape after expansion and filling with contrast reagent resembles the shape of a roller of a “dog-bone” type , whose middle section has a necking in reference to broader sections at both bases of the roller. The characteristic necking after expansion of the balloon in its middle section allows to locate an implant in this place, particularly a metal frame of the prosthetic heart valve and its immobilization, which prevents and precludes sliding of the implant from the balloon surface. The guiding balloon 1 can also be devoid of the necking in the middle section. However, it constitutes one of the elements of good positioning of the prosthetic heart valve. In its distal part there is a supply line—a catheter 2 made of polyamide with the diameter of 7 Fr (where the abbreviation Fr applied in the field of technology is a unit of length and it is equal to 0.3 mm). In the proximal section of the balloon there is the end of the pipe 3 formed in the shape of a coil called “pig tail”, made of polyamide. The said end is preceded by a special cone (nozzle) whose widened part is aimed at the balloon. Inside the balloon the cone 4 has a necking 5 led out axially from its base, and the said necking is bigger than the diameter of the inner guiding canal 5 and it is connected with canal 6. A similar protrusion 5′ of the canal 6 is made on the other side of the balloon. The said elements 5,5′ additionally prevent sliding of the implant located on the balloon after crimping the said implant in the necking of the balloon. One “pig end” of the pipe 3 is straight and in the final section it is characteristically curled up as a coil. The diameter of the circle from which the said end curled up in a characteristic and unique way is formed is usually 26 mm. However, this diameter fluctuates in the range from 21 to 31 mm. The distance of the outer part of the said curled up end to the straight part is 24.5 mm.The diameter of the pipe 3 is from 4Fr to 6Fr and its whole length from 40 to 60 mm (in FIG. 2 the example of the length 50 mm ad the diameter of 4Fr is presented). The end of the pipe 3 as an atraumatic element protects the left heart ventricle against damage. The length of the balloon between the pipe 3 and the line 2, supplying the contrast reagent is 90 mm and its diameter in the necking is 23 mm. However, it is permissible to change the size in the range from 60 to 110 mm in case of its length and 13 to 33 mm in case of the diameter. The balloon diameter outside the necking is bigger by 2 mm in comparison to the diameter of the necking. The said necking can be made of a thickened material, which lowers the risk of perforation, of the balloon bursting during crimping, and thereafter during expansion of the valve. The proximal and distal sections widen in comparison to the middle section forming an angle of the value in the range of 100-130°. Inside the middle part of the balloon there are 7 platinum—iridium markers located on the special line connecting both ends of the balloon. Their aim is to expose the balloon in the X-rays while maneuvering it during the delivery and implantation process. Moreover, the said markers after tightening the implant on the crimped balloon-constitute an additional anchoring mechanism of the crimped prosthetic heart valve on its surface preventing sliding of the said valve. A special tube-shaped shield 8 with the diameter slightly bigger than the diameter of the catheter 2 is placed on the proximal end of the line 2 (catheter) supplying the contrast reagent to the balloon, which enables maneuvering the system inside the vessel. At the proximal end in the contact point with the distal end of the balloon the shield 8 has a sheath (extension of the tube) which additionally protects a placed implant, particularly a crimped prosthetic heart valve against sliding and against damage. At the other end of the line 8 there is a cap 9 for supplying contrast in the direction of the balloon. At the end of the catheter 2 there is a straight cap with a lateral canal 10 to which a pressure syringe for pumping the balloon is attached. The outer shield 8 with the sheath is removed after delivering the system into the final destination.

The order of expansion of the balloon and filling it with the contrast reagent is characteristic for the system. In the first place the fluid fills the distal section, thereafter the proximal section is filled and finally the middle section, where the crimped prosthetic heart valve, the implant or another device for treating the valve system defects is placed.

Facilitation of the process of the prosthetic heart valve positioning is characteristic and unique for the system. Additionally, there is a possibility of repeat locating and positioning (refixing) the prosthetic heart valve with use of the delivery system inside the vessel as well as of the refixing the prosthetic heart valve which is dislocated.

1. A delivery system for implants used in structural heart diseases by a minimally invasive method comprising a catheter (2) connected with the distal section of the balloon (1) and on the other side with the straight cap with the lateral canal (10) to which a pressure syringe for pumping the balloon is attached, and the catheter is covered from the outside with a special shield (8), and the balloon (1) has a necking in the middle section and inside the balloon and in the catheter (8) there is a canal (6) led outside the balloon formed at the end in the shape of a coil (3) characterised by the fact that inside the balloon (1) or outside it at its distal and proximal ends the system is equipped with at least one anchoring mechanism shapely formed crimped on the balloon surface of the implant preventing its uncontrolled moving.

2. The system of claim 1 characterised by the fact that the anchoring system is placed on the straight section of the canal (3) at the intersection with the proximal part of the balloon (1) and it is formed in the shape of a cone (4) whose widened part is aimed at the balloon.

3. The system of claim 1 characterised by the fact that the anchoring mechanisms are made inside the balloon in its distal and proximal sections in the form of a protrusion (5,5′) of the canal inside the balloon (6).

4. The system of claim 1 characterised by the fact that the anchoring system is made at the proximal end of the shield (8) of the catheter (2) in the form of a sheath getting bigger in the direction of the diameter outlet.

5. The system of claim 1, characterised by the fact that the anchoring mechanisms in the form of markers (8) placed on the outside surface of the canal (6) whose diameter is bigger than the diameter of the canal.

6. The system of claim 1 characterised by the fact that the anchoring systems in the form of a sheath at the end of the shield (8) formed like a cone dilation in the direction of the distal end of the balloon.
